(19) United States
(12) Patent Application Publication
(10) Pub. No.: US 2007/0122307 A1
(43) Pub. Date: May 31, 2007

(54) "Process for eliminating/reducing compounds with a musty taste/odour in materials that are to come into contact with foodstuffs and in foods or drinks"

(75) Inventors: Luis Manuel Da Costa Cabral E Gil, Carcavelos (PT); Carlos Rodrigues Pereira, Amadora (PT)

Correspondence Address: SUGHRUE MION, PLLC, 2100 PENNSYLVANIA AVENUE, N.W., SUITE 800, WASHINGTON, DC, 20037, US

(73) Assignee: CHIP-CENTRO DE HIGIENIZACAO POR IONIZACAO DE PRODUTOS, Sacavem (PT)

(21) Appl. No.: 10/566,582

(22) PCT Filed: Aug. 5, 2004
(86) PCT No.: PCT/PT04/00021

§ 371 Date: Jul. 19, 2006

(30) Foreign Application Priority Data
Aug. 7, 2003 (PT) PT103 006

(51) Int. Cl. A61L 2/08; A23L 3/26

(52) U.S. Cl. 422/22; 426/234

(57) ABSTRACT

The present invention relates to a method for eliminating/reducing compounds that have a musty taste/odor in materials that are to come into contact with foodstuffs and in foods or drinks, based on the reduction/elimination of TCA (2,4,6-trichloranisol in materials that are to come into contact with foodstuffs and in foods or drinks, in particular cork stoppers. The present invention also relates to the products treated with this method. The method of the invention is based on the irradiation with gamma rays of the abovementioned products with an intensity and duration (radiation dose) that causes the molecular degradation of the TCA molecule, thus eliminating or reducing this compound to a level below the detection limit for consumers.

The present invention relates to a method for eliminating/reducing compounds that have a musty taste/odour in materials that are to come into contact with foodstuffs and in foods or drinks, based on the reduction/elimination of TCA (2,4,6-trichloranisole) in materials that are to come into contact with foodstuffs and in foods or drinks, in particular cork stoppers. The present invention also relates to the products treated with this method.

The method of the invention is based on the irradiation with gamma rays of the abovementioned products with an intensity and duration (radiation dose) that causes the molecular degradation of the TCA molecule, thus eliminating or reducing this compound to a level below the detection limit for consumers.

### SPECIFICATION

The present invention relates to a method for reducing/eliminating the main compound responsible for the musty taste/odour of certain foodstuffs, especially wines, sometimes known as “cork taint” in the case of cork stoppers, and it can also be applied to cork slabs, cork granulate and other materials that come into contact with foodstuffs. This invention can further be applied to foods and drinks and materials that come into contact with them, where the abovementioned “musty taste/odour” is present. The present invention also relates to all the products and materials treated using this method.

The method is based on the molecular degradation of 2,4,6-trichloranisole (2,4,6-TCA), hereinafter referred to as “TCA”, which is the main compound responsible for what is known as the “cork taint” in wines (>80%) and the “musty odour” of other products, by irradiating the products (for example cork stoppers) with gamma rays with sufficient intensity and duration in order to eliminate/reduce this compound to lower levels than the detection limit for consumers.

There are various types of cork stoppers: corks comprising a single piece of natural cork and with two or four parts glued together, simple agglomerate corks and “technical” corks consisting of an agglomerate “body” or “head” with natural cork discs (for example champagne corks, “1+1” corks). The processing of these corks involves various operations (see for example Gil, L., Cortiça: Produção, Tecnologia e Aplicação, Ed. INETI, Lisbon, 1998, or Gil, L., A Rolha de Cortiça e a sua Relação con o Vinho, Ed. APAFNA, Portalegre, 2002, which are indicated here as references).

Some cork stoppers, the ones that are marked and treated, also undergo sterilisation using chemical agents and are sometimes packed in waterproof bags (generally containing between 1000 and 1500 corks) that have been previously vacuumed, usually in a gaseous sulphur dioxide atmosphere, where the use of the correct doses (for example 0.65-1 mg/cork stopper) has not caused any problems and which drastically reduces superficial fungal contamination.

The objective of the sterilisation processes existing in prior art is to destroy the microorganisms (microflora) existing in the cork stoppers, by sterilising them. As some of these microorganisms may give rise to metabolites which, with chlorine, may form TCA type chlorine compounds, the reduction/elimination thereof may contribute to reducing the “cork taint”, but they do not work if the TCA has already been formed.

As can been seen from the above, cork stoppers, both natural corks and also agglomerate or technical corks, often have significant levels of microbial contamination. Even though they may undergo certain treatments during production to avoid this contamination, in practice contamination may occur due to the presence of microbial contaminants in the atmosphere. Some of the methods mentioned have not proven to be completely successful and very often the fault lies with the users, since cork stoppers should be used immediately after they have been unpacked and in non-contaminated atmospheres.

All these sterilisation methods tend to reduce the likelihood of taint problems arising. These and other technical aspects relating to “taints” presumably caused by corks and transmitted to wines are explained in the following two references: Gil, L, “A cortiça e o vinho”, Vinhos & Bebidas, No. 18, 2001, p. 44-58, and Gil, L., A rolha de cortiça e a sua relação com o vinho, Ed. APAFNA, Portalegre, 2002. These references also cite cases of “musty tastes and odours” in meat, fruit and even water.

The problem concerning “taints” is complex. These “taints” are described in various ways, for example as being caused by damp paper, chemical products, mouldy wood, etc. Organised campaigns have even been responsible for the decline of certain markets, an increase in the number of rejected corks and an anti-cork “climate”. In any case, it is a serious problem, particularly in the case of champagne, regarding which it was estimated a few years ago that the percentage of bottles affected by this defect was between 0.5 and 2%, which for an annual production of 200 million bottles corresponds to 1 to 4 million bottles. However, these problems have been decreasing and a recent bibliography states that much less than 1% of corks are affected by this taint, in some cases where the wines have been bottled for a long time.

There are numerous references to the intensity of the taints in ordinary wines, namely “cork taint”, some being more exaggerated than others and varying between 1% and 8%. For example, at a tasting session during Wine Challenge 1996 (www.winespectator.com), where 6000 bottles were tasted, 6.3% had problems. A veteran wine taster said that the figure stood at 8% a few years ago and is currently around 2% to 4%, although there are some very recent references which quote a figure as low as 0.3%. It was estimated a few years ago that the cost of the 2% of wasted wine was around 560 million ∈ per year in Europe. On a worldwide level the figure was estimated at several billion US dollars. As can be seen, this is a problem of economic importance, due to the improvement in the quality of the wines produced, which are more refined and stop “masking” contaminants, as well as better knowledge and greater discernment on the part of consumers.

Although the producers of cork use various sterilisation methods during the processing and packaging of cork stoppers, as mentioned above, some secondary contamination may occur. Sterilised corks may be mixed with contaminated corks or there may even be contamination of the flooring or wooden pallets used during transportation or of the surrounding atmosphere. When mould encounters favourable temperature and humidity conditions, it starts to develop and the by-products of its development may form derivatives of TCA, which is the main tainting agent.

TCA is cited as being involved in most cases of wine tainting and it is therefore considered as being the main cause of this type of problem. In order to avoid the problem of TCA, various technological processes are currently being developed or are already being used in cork manufacturing plants, for example:
a) method for removing TCA by means of the removal of volatiles and filtratio;
b) ozone treatment;
c) hydrodynamic extraction of discs and corks (submerging and submitting them to various pressures→expansion—contraction→washing/“rinsing”);
d) application of a protective layer/barrier;
e) high pressure washing in order to minimise polyphenols;
f) ultrasound washing;
g) steam and/or heat (160° C.);
h) autoclave treatment for 18-20 minutes at 130° C., 180 kPa.
i) extraction with supercritical CO₂.

However, as mentioned above, these methods and the sterilisation methods previously described are preventive and contamination of the products may occur at a later stage.

Specifically with regard to TCA, below is a more detailed description of the prior art relating to the identification of this compound and methods of detection and elimination.

In 1982, Tanner, Buser and Zanier identified TCA as the main component responsible for causing a musty odour, being detectable at concentrations of up to 10 ppt (parts per trillion). Rigaud identified around 50 volatile cork compounds, some of which were possibly related to the problems of tainting. Peña-Neira et al. (A. Peña-Neira, B. Fernández de Simón, M. C. García-Vallejo, T. Hernández, E. Cadahía and J. A. Suarez, “Presence of cork-taint responsible compounds in wines and their cork stoppers”, EUR Food Res Technol, 211 (2000) 257-261) noted that for low levels of TCA, the presence of guayacol and pentachlorophenols also has some influence on cork contamination.

Although it is the main compound responsible for tainting wine, TCA is neither toxic nor dangerous to humans in the concentrations normally present in wine (parts per million), but it reduces the quality of the product to be consumed.

TCA can have various different origins: it may be of purely biological origin (synthesis by microorganisms in the presence of chlorine and hypochlorites) or of chemical origin (from chlorophenols through microbial methylation). The sources of chlorine are very varied (atmospheric chlorine, chlorinated products, traditional washing, etc.) and may or may not be associated with the processing of cork slabs.

The detection limit for TCA is 1 ng/l, which is extremely low. It is one of the most powerful naturally occurring aromatic compounds and is normally considered as being undetectable at a level lower than 5 ppt. In white and sparkling wines, this compound can be detected at levels of 2 parts per trillion, which corresponds to one soup spoon in around 2000 Olympic size swimming pools or 1 second in 32,000 years.

The most commonly used technique for analysing wine taints is “Solid-Phase Microextraction (SPME)” coupled to one or more analytical techniques, often gas chromatography/mass spectrometry (T. Evans, C. Butzke and S. Ebeler, “Analysis of 2,4,6-trichloroanisole in wines using solid-phase microextraction coupled to gas chromatography-mass spectrometry”, Elsevier Science, Journal of Chromatography A, 786 (1997) 293-298; M. Mestres, M. Marti, M. Miracle, C. Sala, O. Busto, J. Guasch, Tec. Lab., Publica AS. (Spanish), 22(251) (2000) 289-295).

TCA is difficult to remove mainly due to its low volatility (boiling point=240° C.) and also due to the intrinsic characteristics of cork: impermeability to gas and liquid, thermal and electric insulator and acoustic and vibration absorber. Since the identification of the main cause of wine tainting, TCA, various methods have been published with the aim of eliminating this problem in cork stoppers.

The published methods for eliminating TCA use various different processes: chemical, physical, physicochemical and biological processes. Some focus on eliminating the causes—elimination of the microorganisms present and/or the presence of chlorinated agents, while other methods act directly on the levels of TCA present in the cork.

Following the identification of TCA in 1982 as being the main cause of musty odours, it was two years later that Zehnder et al. (H. J. Zehnder, H. R. Buser, H. Tanner, “Cork Taint Formation in Wine and Its Prevention by an Irradiation Treatment of the Corks”, Deutsche Lebensmittel-Rundschau, 80 (7) (1984) 204-207) published a study on the irradiation of cork stoppers with the aim of preventing the conversion of 2,4,6-trichlorophenol into 2,4,6-trichloranisole by microorganisms using biomethylation (sterilisation). One of the biggest disadvantages of this technique is that it does not remove the TCA found in the internal structure of the cork and the technique focuses on the reduction of microbial contamination. By reducing microbiological contamination, the likelihood of TCA forming is also reduced, meaning that this is an indirect method for its reduction.

A little later Botelho et al. (M. L. Botelho, E. Almeida-Vara, R. Tenreiro and M. E. Andrade, “Searching for a strategy to Gamma-Sterilize Portuguese Cork Stoppers—Preliminary Studies on Bioburden, Radioresistance and Sterility assurance Level”, Radiation Physics and Chemistry, 31(4-6) (1988) 775-781) conducted a preliminary study on the use of gamma radiation in the sterilisation of cork stoppers. This study aimed at determining the gamma radiation resistance of the various microorganisms present in the corks, as well as the level of sterilisation that can be obtained using this technique. Mould was the main contaminant found in the samples studied. With doses of 15 kGy, the level of sterilisation that can be achieved is equivalent to the probability of finding one non-sterilised cork in ten thousand. The process consists of using a radioactive isotope (Cobalt 60) installed in a suitable geometric system (irradiator) and insulated from the outside. The products to be treated are placed inside containers which follow a path close to the irradiator or source, receiving the dose necessary for achieving the desired effect. These authors showed that sterilisation by means of exposure to gamma radiation is an effective and simple technique in the sterilisation process. No other in-depth studies were conducted, namely studies relating to the behaviour of TCA or to the quality of the cork stoppers after treatment.

Sterilisation can also be carried out in an ethylene oxide atmosphere, with exposure for a certain period of time to ultraviolet radiation at the time of bottling. Another possibility for sterilisation is to use ionising radiation. These methods may reduce the formation of TCA, but they do not eliminate it if it has already been formed.

A method for sterilising cork stoppers has also been studied whereby they are exposed to an antibiotic (natamycin or antimycin) contained in an emulsion and applied in a rotary drum (German patent no. 3035646, filed on 20^{th} Sep. 1980). Cork stoppers treated using this method were stored in bags for 6 months. They were then tested in bottles over a period of 2 years with positive results. This is a method which is apparently effective for resolving the microbial problem, but it does not present any solution for removing the TCA found inside the structure of the cork.

Another process cited recommends the use of ozonised water or an ozonised silicone emulsion (1 mg O₃/l, T<30° C.), without the inconvenience of destroying the surface of the corks (Portuguese patent no. 86782, filed on 18^{th} Feb. 1988). The disadvantages are the same as the ones mentioned above.

Deodorisation by heating the cork: in this method the cork is heated to 80° C. for 6 to 8 hours, after which the substances that cause the odours are totally or partially evaporated. However, the problem is that TCA is specifically adsorbed by the macromolecular compounds which constitute the cork, such as cellulose, lignin and suberin, and is difficult to dry desorb by evaporation. Another problem is related to the fact that cork is a good thermal insulator, which requires treatment at a very high temperature in order to reach the desired temperature inside the cork. The heating of the cork to a high outside temperature deteriorates the characteristics of the cork and causes its superficial retraction, concentrating the existing compounds on the inside.

Deodorisation using citric acid: the cork is placed in a 3% volume citric acid solution for 3 to 5 minutes. This deodorisation effect lasts for a short period of time in view of the fact that, due to the low liquid adsorption capacity of the cork, the citric acid solution only reaches a layer close to the surface. After the treatment, the 2,4,6-TCA present in the innermost layer of the cork may migrate towards the outside, causing the same odour.

Deodorisation by oxidation with an alkaline solution of hydrogen peroxide: Portuguese patent no. 89361 (filed on 29^{th} Dec. 1988) describes a process for bleaching and sterilising cork articles by treating them with an alkaline hydrogen peroxide solution (10-300 g of hydrogen peroxide per litre of solution) up to an impregnation rate of 0.05 g H₂O₂/100 g of cork. The treated cork is dried out by subjecting it to an ultraviolet radiation source for a minimum of 2 hours at wavelengths that vary between 200 and 350 nm. The problems of this method are identical to those of the method that uses a citric acid solution.

Deodorisation by contact with ethanol vapours: the cork is placed in an ethanol atmosphere, at a temperature of between 18 and 24° C., for 1 month. Once again, we have the same inconveniences as for the method that uses citric acid.

Deodorisation by n-pentane extraction: n-pentane extraction in Soxhlet is another of the methods cited for removing the TCA present in cork. This method is fairly effective and removes all the TCA present on the outside and inside surface of the cork. It is a method used in laboratory analysis for determining TCA levels. However, as an industrial technique it is extremely expensive and involves a few risks, in terms of both the handling of alcanes and possible contamination of the cork.

Deodorisation using hot water: cork in granulated form is washed in hot water at a temperature of 60° C. This procedure has to be repeated two more times but, due to the affinity of the TCA, it migrates towards the inside of the cork which the water cannot reach, thereby restricting the treatment to the surface, added to which this method is not industrially viable.

Ozone deodorisation/sterilisation: Portuguese patent no. 86782 (filed on 18^{th} Feb. 1988) describes the use of ozonised water or an ozonised silicone emulsion as treatment for the deodorisation/sterilisation of cork. This treatment is carried out at a temperature below 30° C. and the ozone concentration should not be lower than 1 mg/L of water of emulsion. The inconveniences already mentioned for the methods based on the diffusion of solutions into the structure of the cork also apply in this case.

A method for deodorisation of cork, patented by Konishi et al. (I. Konishi, R. Tajima, T. Tsutsumi, U.S. Pat. No. 5,174,956, 1992), uses steam for removing the compounds responsible for tastes and odours, namely TCA. The steam is applied to cork slabs inside a container with controlled pressure (equal to or greater than 1 atm) and temperature (equal to or greater than 100° C.). It has the disadvantages of the similar methods mentioned above.

With the aim of removing TCA from inside the structure of cork, more specifically from cork stoppers, a cork-producing company (RELVAS) and another company engaged in the production of wine-making equipment (VINIPAL), both of which are Portuguese, designed and produced a prototype for the implementation of a treatment for cork disks—a rotary autoclave—in order to eliminate the cork taint from wine by trying to remove the TCA from the corks. The treatment includes the immersion of the corks in ethanol and in a sulphurous solution (sterilisant), followed by a final drying process.

Another process currently exists, known as DELFIN (Direct Environmental Load Focused INactivation), which is aimed at eliminating “cork taint”, and it is already used by one company in the industry (JFS). Whereas the traditional systems only heat the surface of the corks, the new method, which is based on the principle of microwaves, allows the electromagnetic waves to penetrate the cork and heat it up, as well as the microorganisms that are present therein, by heating the water existing inside both the microorganisms and the cork, thus killing the microorganisms and causing the chemical contaminants and foul odours to evaporate. This process is applied to both finished cork stoppers and cork stoppers after washing. The system comprises a large cylinder with conveyor belts for the corks and volatile extraction systems, with various magnetrons (800 W) throughout the whole of the body and a residence time of around 20 minutes, reaching a temperature of about 38° C.

Another industrial process used in the reduction/elimination of TCA is the INOS II process, known as the process of hydrodynamic extraction. This treatment is applied by a producer of cork discs and it consists of bringing the cork discs into contact with hot water inside an autoclave, applying varying pressures (absorption/desorption) and a vacuum for removing the water from inside the cork.

The use of enzymes for inactivating phenols is a solution for reducing/eliminating odours and tastes that is being commercialised by the company NOVOZYMES (www.novozymes.com). Suberase® is a phenol oxidase that polymerises phenols, which thereby do not have any impact in organoleptic terms.

Supercritical extraction is also a technique that has been studied in the removal of TCA from cork stoppers. Taylor et al. (Taylor, Marisa K.; Young, Thomas M.; Butzke, Christian E.; Ebeler, Susan E.; “Supercritical Fluid Extraction of 2,4,6-Trichloroanisole from Cork Stoppers”, J.Agric.Food Chem., 48(6) (2000) 2.208-2211) tested supercritical extraction from cork stoppers using CO₂ and concluded that it is a rapid and quantitative process for extracting TCA from complex solid matrices such as cork.

Pereira et al. (Pereira, C. Silva; Pires, A.; Valle, M. J.; Boas, L. Vilas; Marques, J. J. Figueiredo; Romao, M. V. San, “Role of Chrysonilia sitophila in the quality of cork stoppers for sealing wine bottles”, J. Ind. Microbiol. Biotechnol., 24(4), (2000) 256-261) examined the role of the fungus Chrysonilia sitophila in the quality of cork and concluded that the presence of this fungus in cork prevents the development of odours by not producing the compounds responsible for the musty taste even in the presence of chlorophenols.

A manufacturer of cork stoppers, the company Amorim & Irmãos S.G.P.S., S.A. (www.corkmasters.com/industry/images/advancesinthechemicaldestr.pdf), has described a new process for destroying TCA, known as an “Advanced Oxidation Process”. This process is based on the in situ production of highly reactive hydroxyl radicals from hydrogen peroxide in the presence of ultraviolet radiation. These authors concluded that at least part of the TCA present in the cork is destroyed. The presence in the reactional medium of certain components of the cork prevents the reaction of the free radicals with the 2,4,6-TCA.

International patent application WO 2001041989 A2 (2001) was recently filed for a physicochemical process for removing musty odours and odours in general. This process uses an aqueous suspension of activated carbon obtained from coconut. Washing the cork in this suspension eliminates musty odours as well as other odours.

PCT patent application WO 01/88082 A2 also describes a process for removing odours from food or beverage products by contacting the food or beverage with an aliphatic synthetic polymer film, such as very low molecular weight polyethylene, in order to achieve undetectable concentrations of lower than 5 ppt (parts per trillion).

U.S. Pat. No. 5,353,417 relates to the problem of removing TCA from contaminated corks by steam treating them. U.S. Pat. No. 5,484,620 describes a process which uses polyvinylpyrrolidone and/or polyethylene for filtering beverages and removing polyphenols.

The latter processes are either difficult to apply or are expensive and do not prevent the problem of the subsequent contamination of the treated corks and/or products.

Studies have been conducted on the behaviour of TCA and cork under the influence of radiation—in this case an electron beam. Careri et al. (M. Careri, V. Mazzoleni, M. Musci, R. Molteni, “Study of Electron Beam Irradiation Effects on 2,4,6-Trichloroanisole as a Contaminant of Cork by Gas Chromatography—Mass Spectrometry”, Chromatographia, 53 (9-10) (2001) 553-557) examined the behaviour of TCA solutions under the influence of an electron beam of varying intensities in these presence of cork. The results obtained showed that under the effect of an electron beam, the TCA is degraded to intensities of 25-50 kGy. The degradation products are fundamentally mono and dichloro-anisoles. The high level of degradation of the TCA and the low percentage of by-products, in conjunction with the fact that these by-products are non-toxic, makes it possible to conclude that the technique of irradiation is able to reduce the quantity of TCA in alcoholic solutions of this compound. However, nothing is said about the application of this technique to natural cork stoppers, where other substances may interfere, with different radiation ranges.

Mazzoleni et al. (Mazzoleni, V.; Molteni, R.; Furni, M. D.; Musci, M. “Effect of accelerated electron beam irradiation on cork used for stopper production”, Ind. Bevande, 29 (167), 247-257, 2000) ascertained that the irradiation of cork with an electron beam (10 kGy) controlled various strains of fungi, that irradiation at 1000 kGy reduces the levels of caffeic, cumaric and ferulic (phenolic) acids and that the levels of saturated hydrocarbons increased, having also reported a decrease in chloroanisoles and related compounds, but without formalising a treatment in this field or making any reference to the use of gamma radiation.

German patent DE 10022535 A1 of 29^{th} Nov. 2001, entitled “Reduction of the cork taste/odour in wine and other beverages using electron beam irradiation”, states that the TCA is removed from the cork using this type of radiation, but the experiments are conducted in solution. No reference is made to the use of gamma irradiation.

One manufacturer, the company Advanced Electron Beams (www.advancedelectronbeams.com/), refers to the use of electron beam technology for the destruction of Volatile Organic Compounds (VOCs) and elimination of odours. Another reference (www.iba-sni.com/qe-beam.asp) mentions the use of electron beam technology for sterilising pharmaceutical products.

Cork is very stable to radiation and doses of 1000 kGy produce very small material changes (www.isotron.co.uk/html/iff rcp.htm).

It is well known that gamma irradiation technology and electron beam technology are different, the former being more penetrative and suitable for application to fairly large packing materials, such as cardboard boxes containing bags with cork stoppers or foodstuffs, allowing all the material inside to undergo this treatment. In places where gamma radiation installations already exist, it will not be necessary to build new installations for electron beams.

Douglas W. Cooper (“Reducing Pyrogens in Cleanroom Wiping Materials”, Pharmaceutical Engineering, Jul./Aug. 16 (4) 1996) lists the advantages of gamma irradiation over other alternative methods:
Penetrative power—even in the case of hermetically closed packaging,
Compatibility with different types of products/packaging,
The dose to be applied can be accurately calculated and measured,
It does not leave residues,
Reduction of endotoxin levels.

Gamma rays (http://imagers.gsfc.nasa.gov/ems/gamma.html) are the ones that have the smallest wavelengths and the most energy within the electromagnetic spectrum. Electron beam irradiation (www.organicconsumers.org/irrad/Ebeaminfo.cfmm) uses a high-speed electron projector and nuclear irradiation uses nuclear materials that emit high-speed gamma rays. Electrons can be propelled at higher speeds and can cause more damage to the food than nuclear irradiation. Electron beams penetrate approximately 1 inch and are therefore suitable for flat materials; in other cases (for example bulk cork stoppers), nuclear irradiation is necessary. Unlike nuclear irradiation, electronic beam irradiation may induce a trace amount of radioactivity in the. irradiated materials. It. is-also mentioned (www.iba-sni.com/qe-beam.asp) that most materials behave in a similar way in the presence of two irradiations, but that some materials require an “aeration” period following electron beam irradiation.

Finally, there is also a barrier process where silicone is used as an agent for preventing the migration of the TCA present in the innermost layer of the cork towards the outermost layer, while at the same acting as a barrier to the absorption of the TCA by the cork. U.S. Pat. No. 6,348,243 describes the use of silicone as a coating for the cork in order to prevent the absorption/desorption of TCA. The coating may be applied in a silicone bath, preferably with ultrasonic agitation to improve penetration of the silicone into the cork pores. As a treatment prior to coating, the cork may be subjected to a leaching process using one or more solvents, thereby reducing the concentration of TCA present in the cork.

As far as foodstuffs are concerned, it is well known that packaged foods sometimes have a “musty taste/odour”, presumably also often due to compounds such as TCA. In particular, as well as the above-cited cases of meat, fruit and water, this often occurs, for example, with dried fruits, spices and similar foodstuffs, and references to commercial food irradiation exist (www.sirr.unina.it/bollettino/Anno%204%20N.1/AG41.htm), for example spices, potatoes, onions and garlic (to prevent the formation of “sprouts”), as well as grape musts, minced poultry meat and animal food, for the sterilisation thereof. At present, there are no FDA regulations on the testing of foodstuffs after gamma or electron beam irradiation.

We are now going to give a more detailed description of the invention, which should be considered to be merely illustrative, and any alterations or modifications understood by persons skilled in the art should also be considered to fall within the scope of the invention.

Regarding the processes known in prior art, it was surprisingly discovered that it is possible to totally or partially eliminate or convert the TCA present in cork stoppers and other products contaminated with this compound, using gamma radiation with a dose sufficient to cause the molecular degradation of the TCA molecule and convert it into molecular residues which do not have the same negative organoleptic characteristics. This process, when applied to ready-to-use packaged corks (or foods or drinks), inside the actual sealed packages and without permitting subsequent contamination, guarantees the elimination/reduction of the problem until bottling (or consumption in the case of foods or drinks), which constitutes an enormous advantage in relation to the processes of prior art. The radiation dose may vary, for example, usually 15 to 400 kGy, preferably between 90 and 110 kGy and most preferably 100 kGy.

In the case of cork stoppers sold as a final product (superficially finished, packed in polyethylene bags and placed inside a cardboard box, the number of corks per box being around 5000-6000), they can be treated, packaged and prepared for shipment, in view of the penetration capacity of the gamma radiation and the usual dimensions of these boxes (around 0.5×0.5×0.5 m).

The products to be treated are positioned in a geometric layout close to a gamma radiation source such as a radioactive isotope (for example Cobalt 60), at a certain distance and for a certain period of time, in order to receive the dose necessary for the molecular degradation of the TCA.

As well as natural cork stoppers, this process can also be applied to cork slabs or to cork granulate, i.e. suberous material at an intermediate phase in the production of cork stoppers made of natural cork or technical corks (made of agglomerated cork or agglomerated cork with cork discs). At the same time, this process can also be applied to foodstuffs, preferably packaged products.

Hereunder are a few examples of embodiments of the present invention, which must not be considered as being restrictive but rather as specific examples to facilitate the understanding of the invention.

In each of the following examples, the same procedure was used for determining the presence of TCA in the cork stoppers, with the following methodology:

treated and non-treated cork stoppers were granulated to a grain size of approximately 2 mm. 3 g of the granulated mixture undergo steam distillation until a volume of 250 ml of distillate is obtained, from which 20 ml are removed and poured into a 40 ml glass flask, adding 0.2 ml of internal standard TCA and 3 g of NaCl. In order to extract the chloroanisoles an SPME syringe is used, with 30 minutes exposure in a prechamber. After this exposure time, the compounds retained in the fibre are analysed using GC/MS—SIM, monitoring the following ions: m/z 161, 176, 178—dichloroanisoles; m/z 195, 210 and 212—trichloroanisoles; m/z 231, 244, 246—tetrachloroanisoles; m/z 265, 278, 280—pentachloroanisoles. All the results are obtained by interpolation of the calibration curve. The compounds analysed are:

DCA: dichloroanisole 2,4,6-TCA: 2,4,6-trichloroanisole 2,3,4,6-TeCA: 2,3,4,6-tetrachloroanisole PCA: pentachloroanisole

### EXAMPLE 1

In order to demonstrate the validity and advantages of the present invention, various assays were carried out. The cork stoppers used have a dimension of 44×24 mm and they presented all the obvious signs of the presence of “yellow stain”. It has been proven that there is a link between “yellow stain” and the presence of TCA in cork (results of the Quercus Project).

Batches of 10 cork stoppers to be treated—see hereunder the various types selected—were cut in half, perpendicularly to their length, thus giving two batches of 10 half corks. One of these batches was subjected to gamma irradiation in an irradiator with a cobalt 60 source and with a radiation dose in accordance with the conditions defined in Table 1, and the other halves of each batch were kept inside plastic bags, having been previously wrapped in aluminium foil for the purpose of a subsequent comparative analysis in order to determine the presence of TCA in accordance with the method described above. The treated cork stoppers were also subsequently analysed using the method described above in order to determine the presence of the various chloroanisoles. The results are also given in Table 1, where the references A1, A3, B1, B2, C1, C2, C3 refers to the batches where the cork stoppers displayed “yellow stain”, a defect in the cork which indicates a strong likelihood of TCA contamination.

TABLE 1

| | | | | Chloroanisoles | | | |
|---|---|---|---|---|---|---|---|
| | | | | detected | | | |
| | | | | | 2,4, | | |
| | | | Radiation | | 6- | 2,3,4, | |
| | Date of | Cross | Applied | DCA | TCA | 6-TeCA | PCA |
| Reference | Analysis | References | (kGy) | (ng/g) | (ng/g) | (ng/g) | (ng/g) |
| A1 | 20020529 | | 15.44 | 12.6 | 41.7 | N.D. | 4.2 |
| Control Halves | | | | 12 | 42.3 | N.D. | <1 |
| (not treated) | | | | | | | |
| A3 | 20020529 | | 37.64 | 4.8 | 19.6 | N.D. | <1 |
| Control Halves | | | | 3.3 | 14.1 | N.D. | <1 |
| (not treated) | | | | | | | |
| B1 | 20021018 | | 100 | 7.9 | 8.5 | N.D. | <1 |
| Control Halves | | | | 11.4 | 60.1 | N.D. | <1 |
| (not treated) | | | | | | | |
| B2 | 20021018 | | 400 | 18.4 | 31.4 | N.D. | <1 |
| Control Halves | | | | 12.4 | 65.7 | N.D. | <1 |
| (not treated) | | | | | | | |
| C1 | 20021127 | | 80 | N.D. | 34.6 | N.D. | <1 |
| Control Halves | | | | 3.5 | 23.2 | N.D. | <1 |
| (not treated) | | | | | | | |
| C2 | 20021127 | | 150 | N.D. | 38.6 | N.D. | <1 |
| Control Halves | | | | 22.2 | 90.2 | N.D. | <1 |
| (not treated) | | | | | | | |
| C3 | 20021127 | | 250 | 4.8 | 20.6 | N.D. | <1 |

| | | | | |
|---|---|---|---|---|
| Control Halves | 12.5 | 78.7 | N.D. | <1 |
| (not treated) | | | | |

N.D. - not detected

It was also found that at a dose of over 100 kGy there was a marked reduction in the level of TCA, varying between approximately 86% at 100 kGy and 52% at 400 kGy.

### EXAMPLE 2

Table 2 relates to a new set of assays, carried out in the same way as above, but in this case using batches of 5 whole corks. In this case, the references W1, W2 and W3 designate corks treated with different radiation doses (90, 100 and 110 kGy), all coming from the same batch of contaminated commercial corks designated as W4.

TABLE 2

| | Cross | Radiation | | 2,4,6- | 2,3,4,6- | |
|---|---|---|---|---|---|---|
| Date of | Refer- | Applied | DCA | TCA | TeCA | PCA |
| Analysis | ences | (KGy) | (ng/g) | (ng/g) | (ng/g) | (ng/g) |
| 20030609 | W1 | 90 | — | 14.4 | — | — |
| 20030609 | W2 | 100 | — | 2.9 | — | — |
| 20030609 | W3 | 110 | — | 6.0 | — | — |
| 20030609 | W4 | N.T. | — | 137.2 | — | — |

N.T. - Not treated

Considering the detection limit of 10 ppt mentioned in the bibliography, it can be seen that treatment at 100 and 110 kGy achieved the “elimination” (concentration below or near the detection limit) of the TCA present (initially 137.2 ppt) and even at 90 kGy the level of contamination was reduced by around 90%.

### EXAMPLE 3

Four batches of cork stoppers of 44×24 mm—125 corks per batch—were duly packed in aluminium foil bags in an atmosphere containing sulphur dioxide (SO₂), this being similar in every way to current industrial processes. The four batches were packed in a cardboard box of 40×40×40 cm, duly divided into four parts.

The batches were analysed beforehand by soaking three samples of 25 corks from each batch in a solution of 11% ethanol/89% water in a 11 glass bottle for 24 hours. Table 3 describes the four batches used, as well as the initial levels of 2,4,6-TCA calculated on the basis of the arithmetical average of the results of the three batches analysed.

TABLE 3

| | Cork stopper | 2,4,6-TCA |
|---|---|---|
| Batch | quality class | (ng/l) |
| A | 1^{st}/2^{nd} | 8 |
| B | 1^{st}/2^{nd} | 30 |
| C | 3^{rd}/4^{th} | 7 |
| D | 3^{rd}/4^{th} | 27 |

The cardboard box of 40×40×40 cm containing the four batches was subjected to gamma irradiation with an intensity of 100 KGy. After the assay, the four batches were analysed by removing two samples of 25 corks and proceeding as described above. Table 4 shows the arithmetical average of the levels of 2,4,6-TCA for each batch obtained after treatment.

TABLE 4

| | 2,4,6-TCA |
|---|---|
| Batch | (ng/l) |
| A | <2 |
| B | 5 |
| C | <2 |
| D | 4 |

As may be observed in Table 4, all the levels of 2,4,6-TCA present in the corks were reduced to levels equal to or lower than 5 ng/l, which represents a reduction in all cases of more than 80% in relation to the initial figure (Table 3). In some cases (A and C), the levels of 2,4,6-TCA obtained after treatment are lower than the detection limit for the analytical method used. However, more importantly, all the levels obtained are lower than the organoleptic detection limits that are currently considered.

1. Process for eliminating/reducing compounds with a musty taste/odour in materials that are to come into contact with foodstuffs and in foods or drinks, characterised in that it relates to the reduction/elimination of 2,4,6-trichloroanisole (TCA) in materials that are-to-come into contact with foodstuffs and in foods or drinks, based on irradiation with gamma rays at a radiation dose in the range of 15 to 400 kGy, preferably between 90 and 110 kGy and most preferably 100 kGy, reducing the concentration of this compound to levels below the detection limits for consumers.

2. Process according to claim 1, characterised in that it is preferably applied to cork stoppers.

3. Process according to claims 1 and 2, characterised in that it is preferably applied to cork stoppers packed as a final product in sealed plastic bags placed inside cardboard boxes.

4. Process according to claims 1 to 3, characterised in that the products to be treated are positioned in a geometric layout close to a gamma irradiation source (for example Co⁶⁰), at a distance and for a period of time sufficient for supplying a radiation dose necessary for the molecular degradation of the TCA.

5. Process according to claim 4, whereby this molecular degradation makes it possible to reduce the concentration of the TCA to levels below the detection limits for consumers.

6. Process according to claims 4 and 5, characterised in that the radiation dose range is 15 to 400 kGy, preferably 90 to 110 kGy and most preferably 100 kGy.

7. Process according to claims 1, 4 and 6, characterised in that this process can also be applied to cork slabs or to cork granulate, i.e. suberous material at an intermediate phase in the production of cork stoppers made of natural cork or technical corks (made of agglomerate cork or agglomerate cork with cork discs),
Process according to claims 1, 4 and 6, characterised in that this process can also be applied to foods and/or drinks, preferably packaged products.

8. Products that are to come into contact with foodstuffs, especially cork or cork stoppers or packaging, and also foods or drinks, treated according to claim 1.

9. Products that are to come into contact with foodstuffs, especially cork or cork stoppers or packaging, and also foods or drinks, treated according to claim 1.
